(19) United States
(12) Patent Application Publication
(10) Pub. No.: US 2009/0254134 A1
(43) Pub. Date: Oct. 8, 2009

(54) Hybrid ultrasound/electrode device for neural stimulation and recording

(75) Inventors: Robert Nikolov, London (CA); Souhile Assaf, London (CA)

Correspondence Address: ROPES & GRAY LLP, PATENT DOCKETING 39/41, ONE INTERNATIONAL PLACE, BOSTON, MA, 02110-2624, US

(73) Assignee: Medtrode Inc., London (CA)

(21) Appl. No.: 12/322,575

(22) Filed: Feb. 4, 2009

Related U.S. Application Data
(60) Provisional application No. 61/063,554, filed on Feb. 4, 2008.

(51) Int. Cl. A61N 1/00; A61B 5/0476; A61B 8/00; A61N 7/00

(52) U.S. Cl. 607/3; 600/544; 600/439

(57) ABSTRACT

An invasive device that uses electrodes in conjunction with ultrasound is used to enhance and localize electrical recordings and stimulation of neurons. The current design is confined to operate within an elongated geometry for ease of insertion and minimal disruption to brain tissue.

### CROSS-REFERENCE TO RELATED APPLICATIONS

This application claims the benefit of priority of U.S. provisional application Ser. No. 61/063,554 filed Feb. 4, 2008, the entirety of which is incorporated herein by reference.

### BACKGROUND OF THE INVENTION

Electrical stimulation and recording of neurons is of significant use for diagnosis and treatment of many neurological disorders. Neural recordings are a measurement of the electric field potential created by neuronal activity. An electrode may be used to acquire this measurement to directly map neuronal activity. Neuronal stimulation may be initiated by introducing an electric field potential of a certain magnitude and frequency. To stimulate a neuron, the magnitude and frequency of the applied field must exceed a biological threshold (rheobase and chronaxie respectively). Neuronal stimulation is useful for neurosurgery and therapeutics, as it provides a means to modify neuronal activity.

The combination of neuronal stimulation and recording provides a useful mechanism for feed back which may be used to form and synchronize therapeutic stimulation in real time on a per subject basis.

The neural stimulation and recording of neurons by an electrode are very local. As a result, the probe used to transmit or receive electric field potentials must be in close proximity to the target neuron(s). For neural disorders involving neurons embedded deep in the brain, deep brain stimulation (DBS) is required.

Some disorders approved for DBS therapy include; Parkinson's, essential tremor, depression etc.

Disorders being studied for DBS include; epilepsy, tourette's syndrome, obsessive compulsive disorders etc.

The location of the electrode within the brain is crucial in targeting specific structures. Many deep brain structures that are candidates as therapeutic sited are small with large patient to patient variability of both location and size. For example, the sub thalamic nucleus (stn) is a common target for Parkinson's disease treatment. The size and location of the stn is highly variable and has an approximate length of 5 mm.

Methods other than ultrasound for electrode localization have included but at not limited to CT, MRI, NIR, the electrode itself receiving neural information characteristic to the neuronal population. Ultrasound presents itself as a relatively inexpensive imaging modality capable of real time imaging (>30 frames/s).

Ultrasound imaging the human brain is difficult due to the very large acoustic impedance to the cranial skull, and even when burr hole is made to facilitate electrode entry, the surface area of exposed brain limits the Ultrasound depth of view to the upper layers of the cortex. As a result, invasive deep brain ultrasound imaging is not a viable imaging modality for deep brain regions. However, in cases where invasive measures are being made for DBS, the introduction of ultrasound as a localizing imaging modality becomes a viable solution.

There have been recent studies showing that acoustic energy can modify the threshold for electrical stimulation. Thus, in addition to ultrasound imaging for localization, the acoustic energy may be used to modify the stimulation of neurons as well.

Further benefits of including ultrasound into a catheter based design for DBS is it's suspected potential for blood brain barrier disruption for drug delivery. The use of this combination may provide a means for more efficient drug delivery. For instances when the drug used is suspected of modifying neuronal activation, a direct measurement of the activation may be recorded and modified.

The benefits of combining ultrasound and electrode therapy into a device which permits ultrasound imaging for localization and drug delivery introduces extensive research and clinical capabilities.

### BRIEF DESCRIPTION OF THE DRAWINGS

FIG. 1 depicts an apparatus and an interface according to an illustrative embodiment of the invention.

FIG. 2A depicts an isometric view of a probe assembly according to an illustrative embodiment of the invention.

FIG. 2B depicts a cross sectional view of a probe body according to an illustrative embodiment of the invention.

FIG. 2C depicts a cross sectional view of a probe tip according to an illustrative embodiment of the invention.

FIG. 3A depicts column organization of elements along a probe body depicting lateral interaction with surrounding tissue.

FIG. 3B depicts radial organization of elements along a probe body for lateral interaction with surrounding tissue.

### DETAILED DESCRIPTION OF THE INVENTION

In the following detail description of the illustrative embodiment of the invention, references are made to the accompanying figures. The design shown in the illustrations is only a specific embodiment in which the invention may be practiced. It is understood that other embodiments may be utilized and structural changes may be made without departing from the scope of the invention as outlined in the claims. Unless stated otherwise, the figures of the drawing are rendered primarily for clarity and thus may not be drawn to scale.

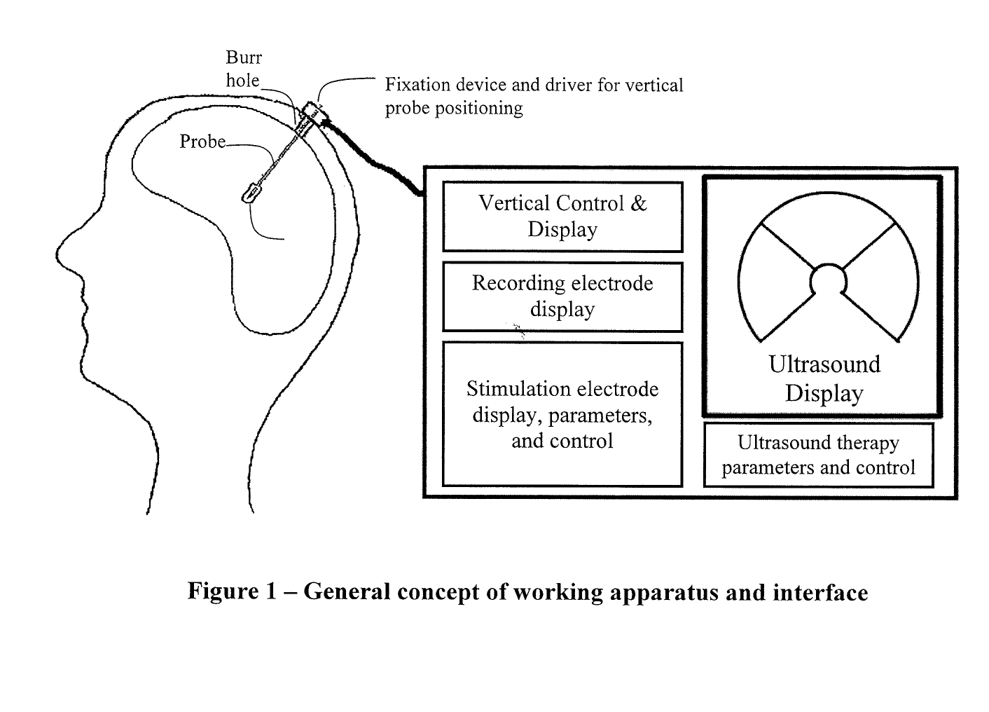

The embodiment of the invention is shown in FIG. 1. In this chosen scenario, access to neural tissue is exposed through a burr hole through the cranial skull. The probe then enters this burr hole and enters the brain. In this example, the depth of probe penetration is electronically controlled (i.e. by a stepper motor) that interfaces to the control user interface display. Since the elongated probe is limited to 1-dimension of control after insertion, a pre imaging scan (MRI, CT, etc) will be required to locate the target of interest to avoid multiple insertions which may cause damage to otherwise healthy tissue.

The control panel and display is shown in FIG. 1 with controls and displays for: electrode stimulation, electrode recording, ultrasound imaging, ultrasound therapy delivery, and probe position.

The ultrasound imaging localization of the probe with respect to the surrounding tissue may be supplemented with the neural recordings along the lateral and distal tip of the probe. The utility of simultaneous used of focused ultrasound with neural stimulation of the probe can also be of use to enhance stimulation of selected targets.

The depth of penetration will be controlled by the user with the ultrasound imaging guidance from multiple dimensions of real-time date acquired by the probe. Since there are many degrees of freedom, the end user will choose what information best suited there needs for target localization. Three B-mode scans are sketched in this embodiment of the invention as an example. The ability of the probe to localize the electrode position within tissue in real time is a major utility of this probe.

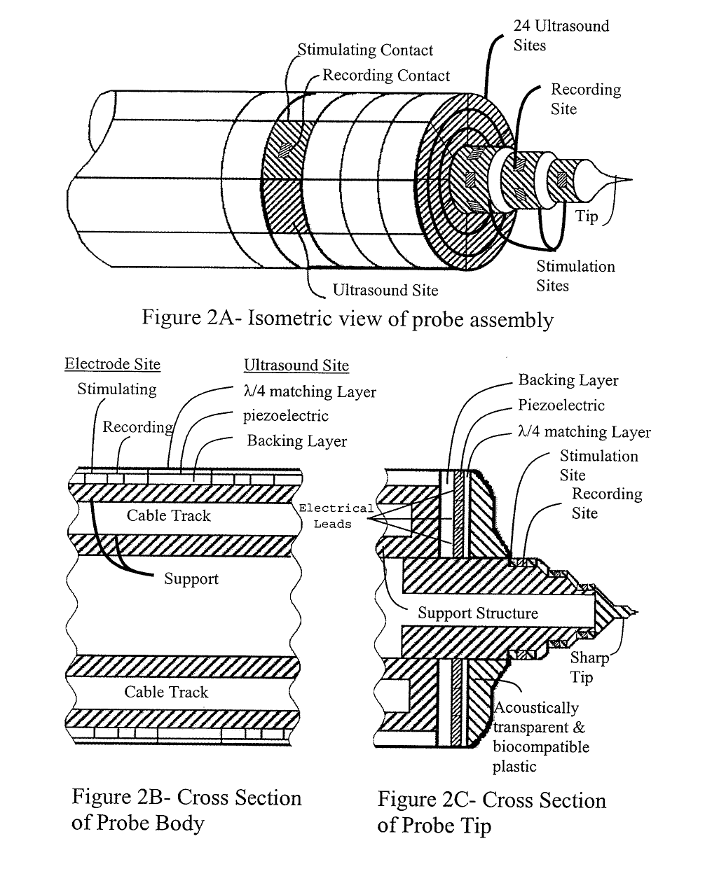

The electrode/ultrasound probe is illustrated in FIG. 2A. The body of the probe assembly may have a stimulating and/or recording electrodes and/or ultrasound transducers, or just insulation material. The probe consists of ultrasound transducers on the shaft (FIG. 2B) and tip (FIG. 2C) to provide lateral and axial imaging or therapy respectively. High power ultrasound therapy will require different materials and mounting as compared to an imaging element. The distribution of these elements may remain a variable for further design consideration.

The ultrasound transducer is shown as a low power imaging design with a quarter wavelength matching layer, a piezoelectric material, and backing substrate. The electrical connections to the shaft and tip (for the electrode and ultrasound transducers) are fed through a wire bundle in the cable track.

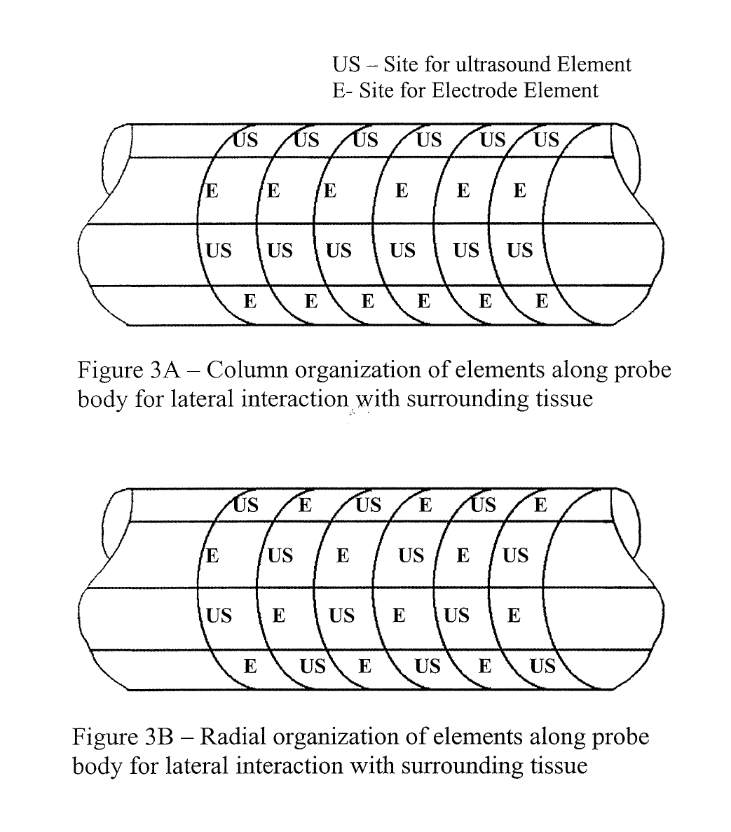

The probe tip has been illustrated with a 24 element segmented annular array to image in 3 spatial dimensions in the axial direction to localize the probe tip within surrounding tissue as shown in FIG. 3C. The electrode tip is not shown in cross section. The electrode tip may be constructed from multiple thin film substrates (as shown) or simply a single insulated metal wire filament with an exposed end. The exposed electrode should have a pointed surface to facilitate entry into the tissue. Furthermore, an acoustically transparent (acoustic impedance approximately equivalent to water or brain tissue.) layer may be used to reduce the exertion force required to penetrate the tissue and to protect the probe itself.

A major constraint is that the diameter of the shaft needs to be approximately 1 mm. The dimensions of this probe are miniature, but still remain in the technological realm of the possible with conventional lithography and micro fabrication and laser machining techniques.

Two distributions of ultrasound and electrode elements are shown in FIGS. 3A and 3B. Although random distributions of these elements may be possible, the functionality and manufacturing considerations tend to promote either the radial or column organization of elements. The periodicity of the elements also reduces the complexity of focusing/steering either the ultrasound beams and/or the stimulation pattern. Although both patterns are shown on a circular cross section, either pattern could also be fabricated with a polygonal cross section.

### REFERENCES

U.S. Pat. No. 7,050,856, issued to Stypulkowski
U.S. Pat. No. 7,047,084, issued to Erickson
U.S. Pat. No. 7,010,356, issued to Jog
U.S. Pat. No. 6,564,079, issued to Cory
U.S. Pat. No. 6,466,822, issued to Pless (2002)
U.S. Pat. No. 6,473,653, issued to Schallhom
U.S. Pat. No. 6,356,784, issued to Lozano et al.
U.S. Pat. No. 6,330,466, issued to Hofmann et al
U.S. Pat. No. 6,304,784, issued to Allee,
U.S. Pat. No. 6,301,492, issued to Zonenshayn,
U.S. Pat. No. 6,249,708, issued to Nelson et al.
U.S. Pat. No. 6,011,996, issued to Gielen (2000)
U.S. Pat. No. 6,095,148, issued to Shastri(2000)
U.S. Pat. No. 5,935,159, issued to Cross, Jr,
U.S. Pat. No. 5,836,875, issued to Webster Jr.
U.S. Pat. No. 5,824,027, issued to Hoffer et al.
U.S. Pat. No. 5,571,148, issued to Loeb
U.S. Pat. No. 5,405,375, issued to Ayers et al.
U.S. Pat. No. 5,215,088, issued to Normann
U.S. Pat. No. 5,095,905, issued to Klepinski
U.S. Pat. No. 4,649,937, issued to DeHaan et al.
U.S. Pat. No. 4,573,481, issued to Bullara
U.S. Pat. No. 4,444,195, issued to Gold.
U.S. Pat. No. 3,955,560, issued to Stein
U.S. Pat. No. 3,774,618, issued to Avery
U.S. Pat. No. 7,037,270 issued to Seward
U.S. Pat. No. 6,969,354 Issued to Marian
U.S. Pat. No. 6,942,677 issued to Nita et al.
U.S. Pat. No. 6,863,653 issued to Zanelli
U.S. Pat. No. 6,024,703 issued to Zanelli et al
U.S. Pat. No. 7,273,458 issued to Prausnitz et al.
U.S. Patent Application No. 20020082527 Liu et al.
U.S. Patent Application No. 20080319375 Hardy
U.S. Patent Application No. 20080046053 Wagner et al
U.S. Patent Application No. 20080045882 Finsterwald
U.S. Patent Application No. 20080033297 Sliwa
J Neurosurg. 2004 March; 100(3):541-6. Determining the position and size of the subthalamic nucleus based on magnetic resonance imaging results in patients with advanced Parkinson disease.

What is claimed is:

1. An invasive catheter for neural stimulation and/or recording apparatus comprising;
an ultrasound transducer or transducer array located at the distal end, along the catheter, and/or external to the tissue for transmitting and/or receiving acoustic energy in the radial and/or lateral directions; and
one or more transmit and/or receive electrodes for stimulation and/or neural recordings located at the distal end or along catheter.

2. The apparatus of claim 1 wherein the ultrasound transducer is used to localize the position of the electrode and catheter within the tissue.

3. The apparatus of claim 1 wherein the ultrasound transducer is used to affect the tissue properties in a region of interest using low intensity focused ultrasound (<500 W/cm²).

4. The apparatus of claim 1 wherein the ultrasound transducer is used to affect the tissue properties in a region of interest to increase and/or reduce neuronal stimulation using low intensity focused ultrasound (<500 W/cm²).

5. The apparatus of claim 1 wherein the ultrasound transducer is used to affect the tissue properties in a region of interest using high intensity focused ultrasound (>500 W/cm²).

6. The apparatus of claim 1 where in the ultrasound transducer is used to affect the tissue properties in a region of interest to increase and/or reduce neuronal stimulation using high intensity focused ultrasound (>500 W/cm²).

7. The apparatus of claim 1 wherein the distal ultrasound transducer is removable.

8. The apparatus of claim 1 wherein the ultrasound transducer remains in vivo for chronic implants.

9. The apparatus of claim 1 wherein the ultrasound transducer and/or electrode is used for delivery of therapy.

10. The apparatus of claim 1 wherein the ultrasound transducer is affixed on the catheter.

11. The apparatus of claim 1 wherein the ultrasound transducer is affixed to the electrode.

12. The apparatus of claim 1 wherein the ultrasound transducer is wireless.

13. The apparatus of claim 1 wherein the ultrasound transducer has electrical wire leads.

14. The apparatus of claim 1 wherein a drug delivering catheter may be incorporated into the probe.

15. The apparatus of claim 1 wherein drug delivery is facilitated acoustic energy disrupting the blood brain barrier or other acoustic mechanisms.

16. The apparatus of claim 1 wherein drug delivery is facilitate through an applied voltage at the electrode (ionophoresis).

17. The apparatus of claim 1 wherein the transmission of acoustic energy is provided by a Micro-Electro-Mechanical Systems (MEMS) actuator.
